(19) United States
(12) Patent Application Publication
(10) Pub. No.: US 2026/0253710 A1
(43) Pub. Date: Aug. 27, 2026

(54) METHODS OF TREATING MENTAL HEALTH ISSUES

(71) Applicant: Yale University, New Haven, CT (US)

(72) Inventor: Amber Childs, New Haven, CT (US)

(21) Appl. No.: 19/489,677

(22) PCT Filed: Jun. 7, 2024
(86) PCT No.: PCT/US2024/032956

§ 371 Date: Dec. 3, 2025

Related U.S. Application Data
(60) Provisional application No. 63/507,061, filed on Jun. 8, 2023.

(51) Int. Cl. G16H 20/70; G16H 10/20

(52) U.S. Cl. CPC G16H 20/70; G16H 10/20

(57) ABSTRACT

Provided are digitally-implemented methods of treating a mental health issue in a subject. The methods include digitally delivering a standardized assessment to the subject; analyzing data collected from the standardized assessment; identifying an evidence-based treatment pathway based upon the analysis of the data; and administering the evidence-based treatment pathway to the subject.

### CROSS-REFERENCE TO RELATED APPLICATIONS

The present application claims priority under 35 U.S.C. § 119(e) to U.S. Provisional Patent Application No. 63/507,061, filed Jun. 8, 2023, which application is incorporated herein by reference in its entirety.

### BACKGROUND OF THE INVENTION

Mental health is the costliest health condition in youth, with $13.9 billion in reported mental health treatment expenditures in 2012. Within the space of a decade (2009-2019) CDC data underscored how youth mental health intensified to a crisis and was declared a national health emergency jointly by key groups, including the American Academy of Pediatrics and the US Surgeon General. However, most youth do not get the evidence-based mental health treatment they need (provider shortages, extended wait times, lack of affordable options), increasing the crisis and costs associated with poor outcomes. Further, when they do get connected to care, rates of treatment dropout are staggering, ranging from 28-70% (DeHaan et al., 2018).

Evidence-based behavioral health treatments are proven to reduce symptoms while reducing costs of poor outcomes. Adding a routine measurement practice strengthens those benefits. Measurement-Based Care (MBC) is the evidence-based practice (EBP) of using patient-reported progress data to aid in clinical decision making throughout the course of psychiatric and behavioral health treatment (Scott & Lewis, 2015). The clinical process of MBC is characterized by the core components of “Collect, Share, Act” which refer to 1) systematic and routine collection of patient-reported progress measures (PROMs), 2) sharing timely feedback with the patient about current scores and trends over time, and 3) acting on these data in the context of the patient's experience and goals for treatment (Barber & Resnick, 2022; Resnick & Hoff, 2019).

Large-scale meta-analytic studies of randomized control trials comparing MBC to usual care show that MBC is linked to faster and better overall treatment outcomes, improved ability to detect deterioration and risk of treatment failure, reduction of treatment drop out, improved patient and provider therapeutic alliance, enhanced patient empowerment, and preliminary evidence of reduction of cost associated with care (de Jong et al., 2021; Fortney et al., 2018; Lewis et al., 2019; Parikh et al., 2020). Beyond the impact on care quality, MBC affects health care organizations accreditation status as well as potential reimbursement from payers. MBC is required by the Joint Commission, and is linked to the value-based care landscape where reimbursement depends on measurable quality rather than volume of services rendered.

However, providers and systems have struggled to effectively implement MBC in routine practice, resulting in loss of potential benefit to patients and ineffective revenue capture for providers. Accordingly, there is a need in the art for articles and methods that improve on existing articles and methods of treating mental health, particularly mental health issues in youth through MBC. The present invention addresses this need.

### SUMMARY OF THE INVENTION

In one aspect, a digitally-implemented method of treating a mental health issue in a subject, the method includes digitally delivering a standardized assessment to the subject; analyzing data collected from the standardized assessment; identifying an evidence-based treatment pathway based upon the analysis of the data; and administering the evidence-based treatment pathway to the subject.

In some embodiments, at least a portion of the method is performed through a digital application. In some embodiments, the subject receives and responds to the standardized assessment through the digital application.

In some embodiments, the standardized assessment is delivered to the subject at set intervals. In some embodiments, the set intervals include at least once per week.

In some embodiments, the step of analyzing the data includes at least one of identifying, guiding the selection of, and measuring one or more measures relating to the mental health issue of the subject. In some embodiments, the step of identifying the evidence-based treatment pathway includes selecting or constructing measurement-based care (MBC) based upon the one or more measures. In some embodiments, the step of identifying the evidence-based treatment pathway includes modifying a measurement-based care (MBC) based upon a change in the one or more measures with respect to previously collected data from the subject.

In some embodiments, the step of analyzing the data includes applying an algorithm to the data. In some embodiments, the algorithm is a machine learning algorithm. In some embodiments, the algorithm detects patterns in the data. In some embodiments, the patterns relate to comparison with at least one of a control data set and previously collected data from the subject.

In some embodiments, the method includes automatically communicating data summaries to at least one of the subject and a care provider. In some embodiments, the method includes automatically communicating alerts to the care provider. In some embodiments, the alerts include at least one of a change in symptoms, escalation of symptoms, or deterioration of the subject.

In some embodiments, the method further includes bidirectional integration with electronic health records.

In some embodiments, the method further includes providing care providers with at least one of psychoeducation and support strategies tailored to the data trends of subjects enrolled in the method.

In some embodiments, the subject is a teenager. In some embodiments, the method is configured specifically to engage teenagers. In some embodiments, the method includes at least one feature selected from gamification-streaks, badges, special offerings, and combinations thereof.

### BRIEF DESCRIPTION OF THE DRAWINGS

FIG. 1 shows a schematic illustrating an overview of a measurement selection and decision-making tool.

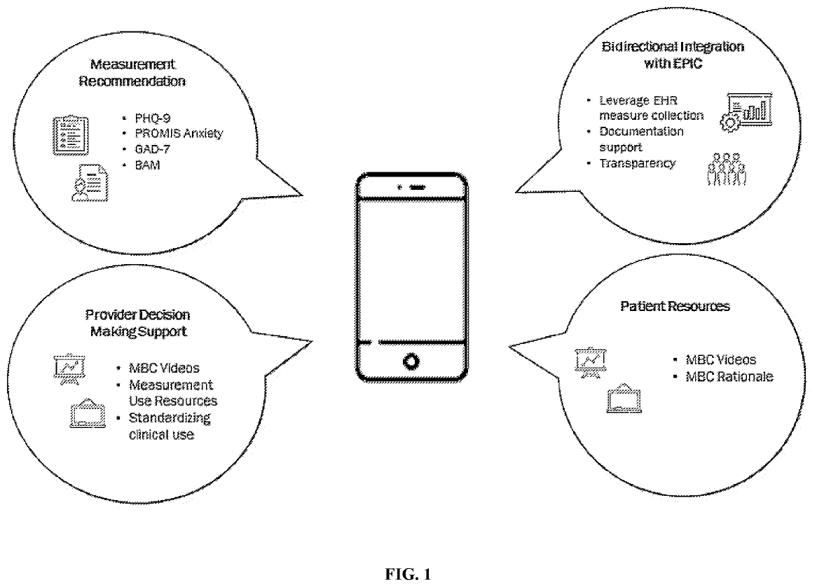

### DETAILED DESCRIPTION OF THE INVENTION

### Definitions

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, the preferred methods and materials are described.

The articles “a” and “an” are used herein to refer to one or to more than one (i.e., to at least one) of the grammatical object of the article. By way of example, “an element” means one element or more than one element.

“About” as used herein when referring to a measurable value such as an amount, a temporal duration, and the like, is meant to encompass variations of ±20% or ±10%, more preferably ±5%, even more preferably ±1%, and still more preferably ±0.1% from the specified value, as such variations are appropriate to perform the disclosed methods.

Ranges: throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

### DETAILED DESCRIPTION

Provided herein are methods of treating mental health issues. In some embodiments, the method includes providing digital, measurement-based care (MBC) to a subject. The subject may include any individual having or suspected of having a mental health issue, including, but not limited to, adolescents, teens, and/or adults. In some embodiments, the method includes delivering a standardized assessment to the subject, analyzing data collected from the standardized assessment, and identifying an evidence-based treatment pathway based upon the analysis of the data. In some embodiments, the method further includes administering the evidence-based treatment pathway to the subject. In some embodiments, at least a portion of the method is administered through a digital application (app).

The standardized assessment may be delivered in any suitable manner and with any suitable frequency. For example, in some embodiments, the standardized assessment is delivered digitally (e.g., through the app or otherwise on a smart phone, tablet, or computer) at one or more set intervals. The set intervals are based upon the subject, stage of treatment, and/or type of mental health issue being assessed/treated. Suitable set intervals include, but are not limited to, every two days, every three days, every four days, every five days, every six days, weekly, bi-weekly, every three weeks, monthly, or any suitable combination, sub-combination, range, or sub-range thereof.

After delivering the standardized assessment, the step of analyzing the data includes identifying, guiding the selection of, and/or measuring one or more measures relating to the mental health issue(s) of the subject. In some embodiments, the analyzing includes detecting patterns in the data. The patterns may be detected in comparison with a control data set, previously collected data from the subject, and/or according to pre-established patterns. In some embodiments, analyzing the data includes applying an algorithm to the data. In some embodiments, the algorithm is a machine learning algorithm. In some embodiments, the algorithm uses evidence-based principals (EBP) to provide autogenerated responses regarding detected patterns in the data. In some embodiments, the algorithm identifies the one or more measures relating to the mental health issue(s) of the subject based upon the patterns in the data. For example, the algorithm may identify and/or guide the selection of one or more measures based upon the autogenerated responses, identify changes in the one or more measures of the subject over time (e.g., between standardized assessments), or a combination thereof.

In some embodiments, the autogenerated responses and/or measures are specific to the mental health issue(s) of the subject. Accordingly, in some embodiments, the autogenerated responses and/or measures can be used to guide the selection of, direct the construction of, and/or provide MBC to the subject. For example, the autogenerated responses and/or measures can be used to support care provider (e.g., pediatrician, primary care provider, or behavioral health provider if available) decision-making, provide decision-making support on clinical use, and/or identify treatment steps/approaches for the subject. The autogenerated responses and/or measures can also be used to monitor progress and/or symptoms. In some embodiments, the method includes adjusting treatment components according to meaningful fluctuations in the reported symptom trends over time and/or aspects of the subject's engagement in other components of the app (e.g., specific interventions, patterns in time of day to access particular coping skills, etc). In some embodiments, the method includes automatically communicating or providing data summaries to the subject and/or care provider. Additionally or alternatively, in some embodiments, the method includes communicating or providing alerts to the care provider. In some embodiments, the alerts include a change in symptoms, escalation of symptoms, and/or deterioration of the subject.

In some embodiments, the method is bidirectionally integrated with EPIC or electronic health records. In such embodiments, the method may provide standardized documentation support and/or solutions to provide guidance and education to patients.

Additionally or alternatively, in some embodiments, the method includes providing caregivers with psychoeducation and/or support strategies tailored to the data trends of the subjects (e.g., youth) enrolled in the app. For example, in some embodiments, the method includes providing caregivers with psychoeducation regarding how depression manifests in youth. In another example, the method includes providing caregivers with support strategies regarding effective communication. The psychoeducation and/or support strategies provided by the method facilitate and/or improve the caregivers ability to effectively support the subject's wellness and treatment.

In some embodiments, the method uses a format and techniques configured specifically to engage teenagers and improve/ensure treatment retention. For example, in some embodiments, the assessments and/or interventions are delivered from a database of expert created content assembled to include novel engagement strategies and techniques designed to retain youth in the treatment. These engagement strategies and techniques include, but are not limited to, gamification-streaks, badges, special offerings/opportunities (e.g., black history month, day of trans visibility, coming out day, etc), or any other suitable strategy/technique for engaging youth. Unlike attempts to re-purpose strategies built for adult audiences, the methods according to one or more of the embodiments disclosed herein are designed with teenagers in mind, leveraging gamification, format, design, and/or engagement strategies that resonate with social media to optimize youth engagement.

The method according to the embodiments disclosed herein provides the subject with evidence-based, regulatory grade digital treatment that uses specific, standardized, and/or guided measurement to deliver personalized brief interventions that fill critical treatment gaps. Without wishing to be bound by theory, it is believed that the methods disclosed herein represent the first measurement feedback system providing clinical decision making guidance so that providers can effectively leverage data points, trends/patterns, and trajectories over time consistent with MBC practice. The methods disclosed herein also leverage the MBC model, providing subjects, providers, and caregivers with data-driven clinical insights. These data-driven clinical insights improve efficiency of care, cost-effectiveness, and/or improve outcomes. Additionally, the methods disclosed herein fill critical gaps in treatment, reducing the need for other more costly interventions linked to symptom deterioration (i.e., psychiatric emergency department use, psychiatric inpatient hospitalizations, intensive outpatient programming, cascading implications for psychosocial deterioration regarding work and education). Furthermore, the methods disclosed herein equip providers with routine data points to ensure optimal reimbursement in the value-based care landscape, where reimbursement depends on measurable impact of care.

Those skilled in the art will recognize, or be able to ascertain using no more than routine experimentation, numerous equivalents to the specific procedures, embodiments, claims, and examples described herein. Such equivalents are considered to be within the scope of this invention and covered by the claims appended hereto.

It is to be understood that wherever values and ranges are provided herein, all values and ranges encompassed by these values and ranges, are meant to be encompassed within the scope of the present invention. Moreover, all values that fall within these ranges, as well as the upper or lower limits of a range of values, are also contemplated by the present application.

The following examples further illustrate aspects of the present invention. However, they are in no way a limitation of the teachings or disclosure of the present invention as set forth herein.

### EXAMPLES

### Example 1

Teenagers need access to evidence-based treatment in a way that works for them and for providers. The marketplace has seen a growth in technology solutions, called measurement feedback systems. Measurement-feedback systems help streamline the process of collecting patient-self reports and summarizing those data. However, this data collection amounts to population screening, not measurement-based care (MBC), and there is no “one-size” fits all measure. As such, these measurement collection and data aggregator solutions do not result in the clinical impact that is possible with MBC.

This Example describes M-Select, an implementation of the methods disclosed herein that provides evidence-based treatment and mental health insights that work for teens and providers, connecting the dots to bring teens, providers and caregivers into sync about mental health. M-Select represents an MBC method that fills known implementation gaps in the current MFS marketplace, so that MBC progress is not hindered.

In behavioral health, a little measurement routine has a big impact. Measurement-Based Care (MBC) uses patient-reported data to guide decision making throughout treatment. MBC is collect, share, act. Collecting patient self-reported progress measures, sharing the results with the patient, and acting on the data in collaboration with the patient to make meaningful changes to care. M-Select is a digital tool that provides measurement selection guidance as well as decision-making support and MBC clinical use tools for providers. M-Select is designed to be seamlessly integrated into usual practice.

For a psychiatrist seeking to use MBC for a patient with depression, there are endless depression measures to choose from. As such, that psychiatrist is realistically treating many different patients, with many different problems, and no measurement guidance in sight. Not only do they need to know what measure to use, they need clinical decision-making tools to ensure standardization and fidelity to practice. Therefore, ultimately, MBC doesn't happen.

To address this, M-Select uses an algorithmic approach to help providers identify the right measure, for the right patient, for the right issue. Then, M-Select gives decision-making support on clinical use to support the cost saving actions. Through bidirectional integration with EPIC or electronic health records, M-Select can also provide standardized documentation support, as well as solutions to provide guidance and education to patients. In contrast to relying on clinical judgement alone, where research shows that clinicians can accurately detect deterioration 21% of the time, MBC through M-Select provides data-driven signals of when a patient is not on track or deteriorating, improving these odds exponentially. Additionally, M-Select gives providers the resources they need to make an impact with MBC, where people drop out less, get better faster, and improve more. Not only does M-Select increase patient collaboration, it has a real impact on the bottom line-as this can circumvent costly psychiatric hospitalizations or emergency department usage.

The disclosures of each and every patent, patent application, and publication cited herein are hereby incorporated herein by reference in their entirety.

While this invention has been disclosed with reference to specific embodiments, it is apparent that other embodiments and variations of this invention may be devised by others skilled in the art without departing from the true spirit and scope of the invention. The appended claims are intended to be construed to include all such embodiments and equivalent variations.

What is claimed is:

1. A digitally-implemented method of treating a mental health issue in a subject, the method comprising:
digitally delivering a standardized assessment to the subject;
analyzing data collected from the standardized assessment;
identifying an evidence-based treatment pathway based upon the analysis of the data; and
administering the evidence-based treatment pathway to the subject.

2. The method of claim 1, wherein at least a portion of the method is performed through a digital application.

3. The method of claim 2, wherein the subject receives and responds to the standardized assessment through the digital application.

4. The method of claim 1, wherein the standardized assessment is delivered to the subject at set intervals.

5. The method of claim 4, wherein the set intervals include at least once per week.

6. The method of claim 1, wherein the step of analyzing the data includes at least one of identifying, guiding the selection of, and measuring one or more measures relating to the mental health issue of the subject.

7. The method of claim 6, wherein the step of identifying the evidence-based treatment pathway includes selecting or constructing measurement-based care (MBC) based upon the one or more measures.

8. The method of claim 6, wherein the step of identifying the evidence-based treatment pathway includes modifying a measurement-based care (MBC) based upon a change in the one or more measures with respect to previously collected data from the subject.

9. The method of claim 1, wherein the step of analyzing the data includes applying an algorithm to the data.

10. The method of claim 9, wherein the algorithm is a machine learning algorithm.

11. The method of claim 9, wherein the algorithm detects patterns in the data.

12. The method of claim 11, wherein the patterns relate to comparison with at least one of a control data set and previously collected data from the subject.

13. The method of claim 1, wherein the method includes automatically communicating data summaries to at least one of the subject and a care provider.

14. The method of claim 1, wherein the method includes automatically communicating alerts to the care provider.

15. The method of claim 14, wherein the alerts include at least one of a change in symptoms, escalation of symptoms, or deterioration of the subject.

16. The method of claim 1, further comprising bidirectional integration with electronic health records.

17. The method of claim 1, further comprising providing care providers with at least one of psychoeducation and support strategies tailored to the data trends of subjects enrolled in the method.

18. The method of claim 1, wherein the subject is a teenager.

19. The method of claim 18, wherein the method is configured specifically to engage teenagers.

20. The method of claim 19, wherein the method includes at least one feature selected from gamification-streaks, badges, special offerings, and combinations thereof.
